(19) United States
(12) Patent Application Publication
(10) Pub. No.: US 2024/0115306 A1
(43) Pub. Date: Apr. 11, 2024

(54) ABLATION PROBE WITH INNER COOLING

(71) Applicant: Erbe Elektromedizin GmbH, Tuebingen (DE)

(72) Inventors: Hanna Andel, Moessingen (DE); Marcus Adler, Burladingen (DE); Felix Karcher, Tuebingen (DE)

(73) Assignee: Erbe Elektromedizin GmbH, Tuebingen (DE)

(21) Appl. No.: 18/377,680

(22) Filed: Oct. 6, 2023

(30) Foreign Application Priority Data
Oct. 7, 2022 (EP) 22200327.9

(51) Int. Cl. A61B 18/14

(52) U.S. Cl. CPC A61B 18/14; A61B 2018/00577

(57) ABSTRACT

An ablation probe according to the invention comprises at least one electrode, which is held on a hose. The ablation probe is provided with a closure piece from which a wire extends over the entire length and through the hose. A fluid supply line is attached to the wire and has lateral openings for cooling the electrode, the wire being closed at the terminal end. An axial alignment of the openings relative to the electrodes can be achieved due to the fixation of the fluid supply line to the wire, and a misplacement can be avoided.

### CROSS-REFERENCE TO RELATED APPLICATION

This application claims priority to European Patent Application No. 22200327.9, filed Oct. 7, 2022, the en-tirety of which is incorporated by reference herein.

The invention refers to an ablation probe, particularly for HF-ablation of biological tissue.

Probes for HF-ablation serve, for example, to devi-talize tissue sections, e.g. for devitalization of tumors or the like embedded in tissue (e.g. lung or liver).

EP 3 788 974 A1 illustrates an ablation probe of the named configuration having two electrodes held on a hose-like base body, wherein the electrodes are to be connected with the output of an electrical generator. The electrodes are arranged in axial distance to one another on the distal end of the hose-like base body, in order to pass current through the tissue and heat it in this manner, if they are pierced into tissue.

It has turned out to be expedient to cool the electrodes in order to avoid drying of the tissue being in contact with the electrodes. For this purpose the lumen enclosed by the hose is supplied with a coolant, so that the electrode temperature is maintained within limits.

This principle is also realized by probes, as they are illustrated in EP 1 181 896 A1, WO 2012/012869 A1 or US 2005/0010201 A1. Further prior art is disclosed by EP 3 323 366 A1, EP 3 769 706 A1, U.S. Pat. No. 6,106,524, WO 99/08633 A, EP 1 432 360 B1, EP 2 768 563 B1, EP 0 754 075 B1, EP 3 763 314 B1, EP 3 437 579 B1 and WO 94/11059 A. In addition, a cry-oprobe is known from DE 10 2008 024 946 A1 that comprises a cryofluid capillary with lateral exit opening.

During treatment of tissue the electrodes shall be cooled so that the tissue being in direct contact with the electrodes does not become high-ohmic due to drying, but remains moist and thus low-ohmic. Therefore, coolant serving for cooling is output at the distal end of the instrument. For this purpose a fluid supply capillary is arranged inside the lumen of the hose that has at least one opening at its distal end via which the cooling fluid exits and cools the distal end of the probe and thus also the electrodes. This configuration is particularly described in EP 3 788 974 A1.

WO 2012/012869 discloses a heart catheter having a fluid supply line, which is arranged in the lumen surrounded by the catheter and comprises multiple lateral output flow openings in the proximity of its distal end. They serve for coolant supply to the distal end of the catheter.

US 2005/0010201 A1 discloses a probe for cold treatment of biological tissue. The probe consists substantially of a hose body at the distal end of which multiple heat conducting elements are arranged extending through the hose wall. In a first variant inside the lumen of the hose a fluid supply line is arranged, which comprises multiple lateral exit openings that direct one fluid flow respectively onto the respective heat conducting element. In another variant multiple fluid capillaries are arranged inside the lumen of the probe, the respective distal exit openings of which are assigned to one heat conducting element respectively.

In order to facilitate piercing of a probe into biological tissue, it is in addition known to provide the distal end of the probe with a, for example bracket-shaped, electrode on the outside, which is connected to an electrical current supply and if activated is effective to cut into tissue.

It is increasingly desired to provide ablation probes as slim as possible, i.e. with low diameter, without limitation of their functionality with regard to the size of the tissue sections to be treated. Diameters of less than 2.5 mm are intended, whereby also the provided electrode surface becomes accordingly smaller. On the other hand, thereby the current density at the electrode surface increases and thus the need for cooling of the electrodes.

Starting therefrom it is the object of the invention to provide an ablation probe with inner cooling, whereby the invention shall particularly guarantee the further de-crease of the probe diameter and on the other hand, the required quality of the ablation treatment.

This object is solved by an ablation probe according to claim 1:

The ablation probe comprises a flexible hose on which at least one or also two or more electrodes are arranged, which are distanced from one another in axial direction of the hose. They are arranged on or nearby the distal end of the hose. Preferably the electrodes thereby extend around the entire circumference of the hose as well as over an axial length (to be measured between its distal and its proximal end) that preferably is a multiple of its diameter. The electrodes are preferably flexible, so that they are able to follow tight lateral curvatures. They can be configured for this purpose in the type of helical springs. Thereby they have a reduced electrical and thermal conductivity in axial direction compared to a metallic sleeve.

The electrodes are connected to electrical lines that extend from the respective electrode up to the proximal end of the hose and can there be connected via suitable connectors with a supplying generator. The supplying generator is preferably configured to provide a high frequency alter-nating voltage and thus to output high frequency current.

The hose surrounds a lumen that extends from its proximal end up to its distal end and is closed there. For example, a closure piece can be provided for this purpose, which is rigidly connected with the hose and closes the lumen thereof. Inside the lumen a fluid supply line is arranged, which extends in distal direction from the proximal end of the hose up to the electrodes. At the proximal end the fluid supply line can be provided with a suitable connection device, in order to allow connection to a cryofluid source arranged, for example, in the supplying apparatus. The supplying apparatus can thus include the cryofluid source for cooling the electrodes and the generator for current supply to the electrodes in one apparatus. Separated configurations are also possible. Such separate apparatus can be connected by a suitable interface, e.g. a BUS, to form an apparatus system.

The fluid supply line is preferably closed at its terminal end, whereby it comprises in the area of the electrodes at least one lateral opening respectively. In this manner the cryofluid is released at the electrodes respectively, i.e. within the respective volume surrounded by the electrode, in order to uniformly cool the electrode. Due to the specific release of the cryofluid at the electrodes, a high cooling power can be achieved locally exactly where a high deviation is required, namely at the electrodes. On the other hand, a too strong cooling of other areas and thus freezing of tissue on the probe is excluded. In doing so, a performance increase of the ablation probe on one hand or a further diameter reduction on the other hand can be achieved without compromising the ablation performance.

In the area of the electrodes on the fluid supply line one or also multiple lateral openings can be provided respectively. Preferably, however, the cross-section surface of all lateral openings in total serving the fluid exit is less than the cross-section surface of the inner channel of the fluid supply line. Due to this measure, the pressure de-crease along the length of the fluid supply line is minimized and substantially concentrated on the lateral openings serving as nozzles. In this manner the cooling effect is very well localized on the area of the electrodes. Particularly, the electrodes can be effectively cooled independent from their heat conductivity.

At its terminal end the fluid supply line can be realized with a closure piece or a stopper of adhesive or another suitable material establishing a rigid connection with the fluid supply line. The fluid supply line is preferably a thin plastic hose, e.g. from PE, PET or PEEK or another suitable material having a low modulus of elasticity. The lateral openings are preferably laser bores having a diameter between 50 μm and 150 μm, preferably 85 μm to 125 μm. The inner diameter of the fluid supply line is preferably 0.4 mm to 0.7 mm, for example 0.6 mm or 0.57 mm. The fluid supply line is thus extremely flexible.

The closure piece for closing the hose supporting the electrodes can consist of an insulating material, e.g. plastic or ceramic, or also of metal. Particularly, it can also consist of a combination of an electrically insulating material and an electrically conductive material. The electrically conductive areas of the closure piece can be used, for example, as electrodes in order to support piercing of the instrument in biological tissue. Preferably the electrically conductive parts of the closure piece project little or do not project from the insulating material. The electrode formed in this manner at the distal end can facilitate piercing of the instrument into biological tissue without damaging it laterally.

Preferably the closure piece is rigidly connected to a tension-resistant wire, which extends from the closure piece through the lumen of the ablation probe up to the proximal end thereof and is anchored there. The wire serves to support tensile forces during removal of the instrument from a patient, particularly from his/her tissue. The wire re-lieves the hose from tensile forces so that the instrument can be safely retrieved in any case, also in case of treatment problems, e.g. due to sticking of the ablation electrodes on the tissue. The traction wire preferably comprises a high restoring force after deformation induced by an exter-nal force, e.g. by bending of the access system.

In the ablation probe according to the invention the fluid supply line is connected to the traction wire at least at one position. Preferably the fluid supply line is thereby connected with the traction wire between the electrodes and thus the (optional) multiple nozzles. Alternatively, it can also be connected to the traction wire in other areas, preferably outside the areas of the electrodes. In doing so, an axial misplacement of the lateral openings of the fluid supply line relative to the electrodes during bending or kinking of the instrument is avoided. This in turn allows safe and reliable handling and the achievement of small bending radii without effecting the function.

The bending of the instrument is also supported in that the electrodes are flexible. For example, they can be formed by a helically wound wire or by a helically slotted sleeve or the like. Other bendable slotted structures, such as sleeves provided with semi-circular slits, can also be used. In addition, functional separation in the traction wire for supporting tensile forces and in the tube geometry for fluid supply finally allows achieving a low bending stiffness compared to a fluid line having sufficient wall thickness for fluid pressure support and material rigidity for support of tensile forces. In the configuration according to the invention (solid) material having high tensile strength and a geometry with low second moment of area can be selected in order to support the tensile force. This is combined with a fluid line consisting of a material with low rigidity (sufficient to support the fluid pressure) and large inner cross-section. The high second moment of area, however, remains harmless, due to the high material flexibility.

Further details of advantageous embodiments of the invention are subject of dependent claims. In the drawing an embodiment of the invention is illustrated. The drawing shows:

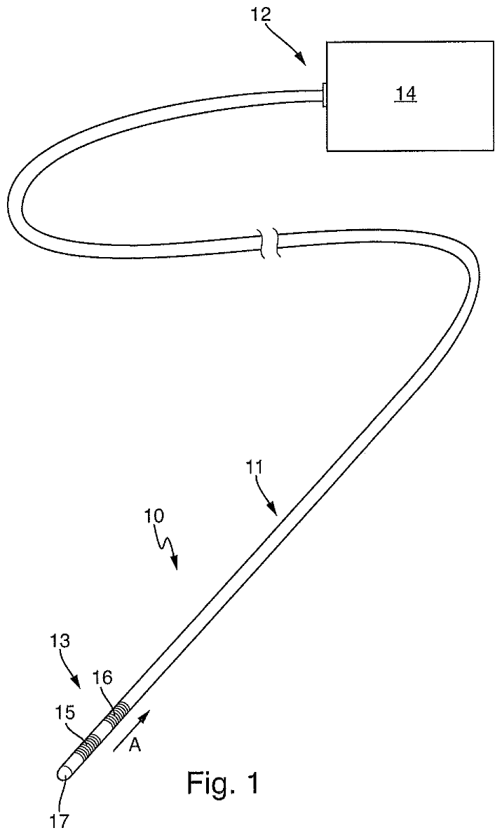

FIG. 1 the ablation probe according to the invention connected to a supplying apparatus in a perspective principle illustration,

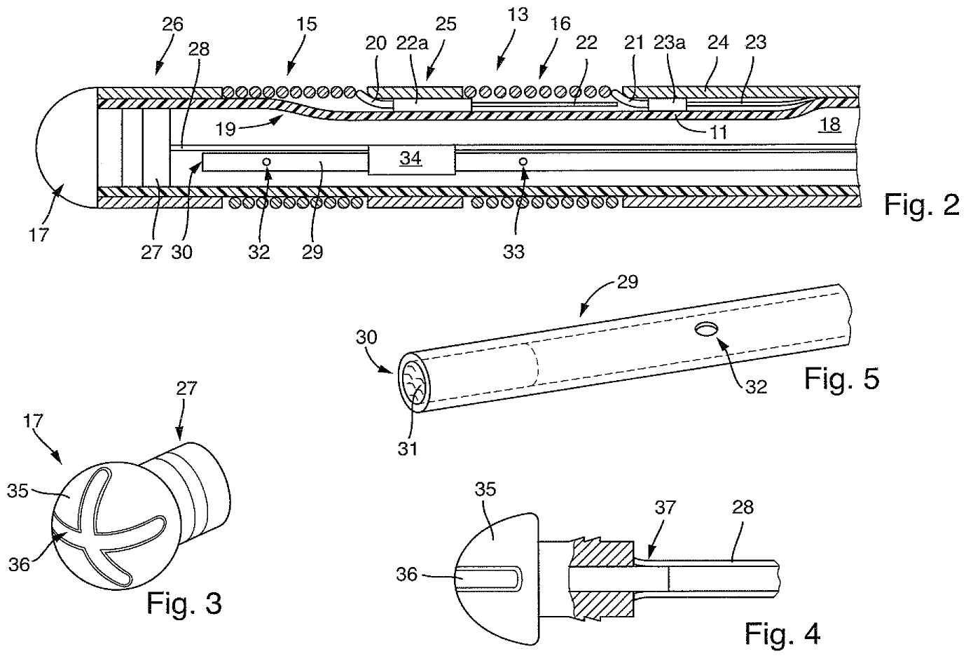

FIG. 2 the distal end of the ablation probe cut in longitudinal direction with view into its lumen,

FIG. 3 an end piece for the ablation probe according to FIG. 1 or 2 in perspective illustration,

FIG. 4 the end piece according to FIG. 3 in partly cut lateral view,

FIG. 5 the distal end of the fluid supply hose arranged inside the lumen of the ablation probe a perspective view.

FIG. 1 discloses an ablation probe 10 usable for the endoscopic use, e.g. the bronchoscopic use, for example for devitalization of lung tumors, but also for other purpos-es. The ablation probe 10 comprises an elongated flexible hose 11 that extends from a proximal end 12 up to a distal end 13 of the ablation probe 10. On the proximal end 12 one or more plugs or another suitable connection device is provided in order to connect the ablation probe 10 with a supplying apparatus 14.

The hose 11 is preferably a flexible plastic hose made of a suitable biocompatible plastic, e.g. PE, PET, PEEK or similar. While its length can have multiple meters, the diameter is in the range of 1 mm to 3 mm. Other dimensions are possible and expedient depending on the application.

The proximal end 13 of ablation probe 10 comprises a first electrode 15 and, at least preferably, a second electrode 16 that are distanced from one another in axial direction A. As required, additional electrodes can be provided that can be supplied with current. The instrument can also have only one single electrode and can be configured as mono-polar instrument. The hose 11 is in addition provided with a closure piece 17 on its distal end that closes its inner lumen 18, apparent from FIG. 2, distally.

The hose 11 encloses this lumen and is thereby configured without gaps and without interruptions. Its wall does not comprise interruptions or through-holes. The electrodes 15, 16 are arranged on the hose 11, particularly in an area in which an oblong depression, e.g. trough 19, is embossed in the wall of hose 11. Therein connections 20, 21 of electrodes 15, 16 are located. From the connections 20, 21 electrical lines 22, 23 extend along hose 11 on the outside in proximal direction up to the end 12 and are there connected to a plug or another connection device for apparatus 14. The lines 22, 23 are connected to the connections 20, 21 by suitable connectors, e.g. crimp barrels 22a, 23a. As illustrated in fig-ure 2, they can be located in direct proximity to the connected electrode 15, 16 respectively or can both be alternatively arranged proximal to the proximal electrode 16.

The lines 22, 23 extend through a gap between hose 11 and a cover hose 24 that is preferably made of a very flexible, slidable plastic. Between the electrodes 15, 16 a sleeve-shaped insulator 25 can be arranged that serves as distance piece between the electrodes 15, 16. In addition, another insulator 26 can be arranged between the distal electrode 15 and the closure piece 17.

The closure piece 17 is preferably rounded at its distal outer surface. For example, it can be configured in hemispherical shape. From the hemispherical head a shank 27 extends into the lumen, whereby the shank 27 is secured to hose 11 in a form-fit manner and/or by an adhesive or other suitable attachment means. In addition, the closure piece 17 is preferably connected with a wire 28 that extends originating from the closure piece up to the force transmitting part of the ablation probe 10 provided, for example, at the proximal end 12 of ablation probe 10, in order to transmit tensile forces from the proximal end reliably to the distal end 13. The force transmitting part of the ablation probe 10 can also be arranged in the course of the length of the ablation probe 10.

Inside lumen 18 a fluid supply line 29 is arranged that can be configured as thin plastic hose, but also as metallic capillary, if applicable. The fluid supply line serves for release of cooling fluid that cools the electrodes 15, 16 and then flows back through cooling lumen 18 to the apparatus 14. It can be released there or at the proximal end 12 of ablation probe 10 into the environment or can also be captured and recycled.

The fluid supply line 29 is closed at its distal end 30. For example, solidified adhesive 31 (FIG. 5) can serve for this purpose, which is located in the distal end section of fluid supply line 29 as stopper.

The fluid supply line 29 comprises a first lateral opening 32 serving as exit nozzle and a second lateral opening 33 also serving as exit nozzle. The first opening 32 serves for cooling the first electrode 15 and is accordingly arranged axially in registration therewith (i.e. within the space surrounded by the first electrode 15). Preferably the opening 32 is thereby arranged slightly further distal than the center of electrode 15. The second lateral opening 33 is arranged inside the space surrounded by second electrode 16. It is therefore arranged in registration with the second electrode 16 and can be located slightly distally to the center of the electrode 16.

The openings 32, 33 are apart therefrom substantially identically configured. As illustrated, they can be arranged in the same radial direction or also offset relative to each other in circumferential direction or they can be different in cross-section.

The following explanation and description of opening 32 in connection with FIGS. 2 and 5 applies, however, accordingly for the opening 33.

While the inner diameter of the fluid supply line 29 can be in the range of approximately half of a millimeter, the diameter of the opening 32 preferably created by laser drilling is only approximately one tenth of a millimeter. In doing so, the nozzle cross-section formed by all openings 32, 33 is remarkably smaller than the free flow cross-section (cross-section area) of the inner diameter and thus the lumen of the fluid supply line 29.

In the presented embodiment to each electrode 15, 16, independent from their number, one nozzle opening 32, 33 is assigned individually in each case. It is, however, also possible to assign two or more openings to each electrode 15, 16 that are offset axially and/or in circumferential direction to one another. Thereby a reliable and uniform cooling of the electrodes is also possible in case of particularly long electrodes 15, 16.

In order to guarantee a safe axial relative positioning of the openings 32, 33 relative to the electrodes 15, 16, the fluid supply line 29 is axially fixated at its distal end. For this purpose it can be connected with the wire 28 that can be made of, for example, spring steel or another material having particular tensile strength, which has preferably a high restoring tendency after deformation, such as for example, nitinol. Also, the use of a plastic wire is possible.

For attachment of the fluid supply line 29 to the wire 28 they can be connected to one another, for example, locally only at one position or also at multiple positions. Particularly, the connection can be arranged between the electrodes 15, 16 and thus between the openings 32, 33. The connection between the wire and the fluid supply line 29 can be realized by a clamp 34, for example, which holds the fluid supply line 29 and the wire 28 together in a friction-fit manner. The clamp 34 can be configured, for example, in the form of a shrinking hose 34 through which the wire 28 as well as the fluid supply line 29 extend and which clamps the wire 28 and the fluid supply line 29 against each other. Instead of a shrinking hose also another suitable connectors can be used, e.g. a crimp barrel of metal, an adhesive joint, a connection by molten and solidified adhesive, a spring clamp or the like.

It is in addition possible to provide a connection between the distal end of fluid supply line 29 and closure piece 17 in addition to or instead of the connection between wire 28 and the fluid supply line 29. For this purpose, for example, an adhesive joint, a crimp joint, a compression joint or the like can be provided. For example, the closed end of the fluid supply hose 29 can be glued into a bore of shank 27. However, it is important in all embodiments that the fluid supply hose 29 is axially immovably fixated particularly in the area of its distal end relative to the electrodes 15, 16. In this manner misplacements of the fluid supply hose and its lateral openings 32, 33 can be avoided re-sulting from a stretching or bending of hose 11 during endoscopic use on the patient.

The end piece 17 is separately apparent from fig-ures 3 and 4. It can be completely made of an insulating material, such as plastic or ceramic, or can also be completely formed of metal, e.g. tungsten carbide. In FIGS. 3 and 4, however, a particular embodiment is illustrated in which the end piece 17 is formed of a metal body 36 and an insulator 35. The metal body 36 is preferably uncovered at the rounded end surface of closure piece 17, whereby it projects slightly or not over the rounded distal end surface of closure piece 17. The insulator 35 can also project slightly distally beyond metal body 36. For example, thereby the metal body 36 can be cross-shaped at its distal end, as illustrated in fig-ure 3. Also other shapes, such as a simple arc or a star having three or more legs, are possible. The metal body 36 extends through insulator 35 and projects, as shown in FIG. 4, from the proximal shank end. There it can be connected with the wire 28 in a tensile-resistant manner, e.g. by a welded joint 37. Alternatively, end piece 17 can also be formed by a metal body on which the insulator is applied in the form of a coating of insulating material.

The wire 28 supporting the tensile forces can be provided with an electrical connection device at the proximal end 12 in order to be supplied with voltage and current from apparatus 14. In doing so, the metal inlay 36 becomes effective as cutting electrode during piercing of the ablation probe 10 in biological tissue.

The ablation probe 10 described so far operates as follows:

The ablation probe 10 is inserted solely or through a respective access instrument, e.g. an endoscope or in case of the ablation of a lung tumor a bronchoscope, into the bronchial tree of the patient. The ablation probe 10 is then moved further in distal direction out of the endoscope toward the tissue that requires treatment, e.g. the lung tumor, and is pierced therein. This can be carried out exclusively me-chanically or, if a distal electrode is present, for example in the form of the inlay 36 with electrical support. For this purpose the electrode formed by the inlay 36 is activated in that electrical energy is supplied thereto via wire 28. The ablation probe 10 is then pierced into the tumor so far until both electrodes 15, 16 are positioned inside the tumor. The distal electrode 36 is inactive subsequently.

Now the electrodes 15 and 16 are applied with treatment voltage and supplied with treatment current. Con-currently or shortly afterwards the fluid supply line 29 is supplied with cooling fluid so that it exits from openings 32, 33. The cooling fluid, e.g. carbon dioxide, is present in the fluid supply line 29 with high pressure of some 10 bar (e.g. 65 bar) and passes through the openings 32, 33 into lumen 18 in which a lower pressure of at most a few bar is present. The openings 32, 33 thereby serve as throttle openings, whereby cold is produced due to the Joule-Thomson-effect. Due to the immovable registration, i.e. axial alignment of the openings 32, 33 with regard to the electrodes 15, 16, the cooling effect is created particularly approximately in the center relative to the electrodes 15, 16 so that a largely uniform cooling of each electrode 15, 16 is guaranteed. Thereby a too extensive heating of the electrodes 15, 16 and thus a drying of the abutting tissue is avoided also if the diameter of the ablation probe 10 is reduced by way of minia-turization down to very small values of, for example, 2.3 mm or less, which results in high current densities at the electrodes 15, 16. With a concept according to the invention, however, an improvement and homogenization of electrode cooling can be effected, whereby the quality of the ablation treatment is increased.

An ablation probe 10 according to the invention comprises at least one electrode 15 and/or 16, which is held on a hose 11. On a terminal end the ablation probe 10 is provided with a closure piece 17 from which a wire 28 extends over the entire length and through the hose 11. To the wire 28 a fluid supply line 29 is attached having lateral openings 32, 33 for cooling the electrodes 15, 16 and being closed at the terminal end. Due to the fixation of the fluid supply line 29 to wire 28, an axial alignment of the openings 32, 33 relative to the electrodes 15, 16 is achieved and a misplacement avoided, which however could result in non-uniformity of cooling of the electrodes 15, 16.

1. An ablation probe comprising:
a flexible hose having at least a first electrode, at least a second electrode, and a distally closed lumen,
a fluid supply line arranged in the lumen and having at least a first lateral opening in the area of the first electrode and at least one second lateral opening in the area of the second electrode.

2. An ablation probe according to claim 1, wherein the fluid supply line is closed at a distal end.

3. An ablation probe according to claim 1, wherein the flexible hose is provided with a closure piece at a distal end.

4. An ablation probe according to claim 3, wherein the closure piece comprises an insulating material or an electrically conductive material.

5. An ablation probe according to claim 3, wherein the closure piece consists of a combination of an electrically non-conductive and an electrically conductive material.

6. An ablation probe according to claim 3, wherein the closure piece consists of an insulator and a metal body, the metal body having an exposed distal portion.

7. An ablation probe according to claim 3, wherein the closure piece comprises a metal body having an insulative coating on portions of the metal body.

8. An ablation probe according to claim 3, wherein the closure piece is connected to a wire that extends through the lumen.

9. An ablation probe according to claim 8, wherein the fluid supply line is connected to the wire.

10. An ablation probe according to claim 9, wherein the wire is formed of a solid material and, when combined with the fluid supply line, comprises a lower bending re-sistance at the same level of tensile strength than a tube that combines the tensile force support and the fluid supply.

11. An ablation probe according to claim 9, wherein the wire and the fluid supply line are connected to each other by a clamp.

12. An ablation probe according to claim 1, wherein the at least first and at least second electrodes are flexible.

13. An ablation probe according to claim 1, wherein the flexible hose is configured without interruptions.

14. An ablation probe according to claim 1, wherein the fluid supply line is a plastic hose.

15. An ablation probe according to claim 1, wherein the first lateral opening and second lateral opening are laser bores.

16. A method for tumor ablation comprising the following steps:
piercing an ablation probe into tissue that comprises an area that requires treatment,
positioning of an at least one electrode coupled to the ablation probe in the tissue that requires treatment,
supplying current to the at least one electrode and con-currently
cooling the at least one electrode with a gaseous fluid that is conducted via a fluid supply line into a lumen surrounded by the at least electrode and is released via a throttle opening into the lumen,
retracting the ablation probe by transmitting a tensile force via the traction wire onto a closure piece) coupled to the ablation probe.

17. The method according to claim 16, further comprising the step of applying an electrical voltage to a metal body during piercing of the ablation probe for cutting tissue located distally in front of the metal body.
